(19) United States
(12) Patent Application Publication
(10) Pub. No.: US 2025/0064848 A1
(43) Pub. Date: Feb. 27, 2025

(54) BIOACTIVE GLASS COMPOSITIONS AND METHODS OF TREATMENT

(71) Applicant: The Curators of the University of Missouri, Columbia, MO (US)

(72) Inventors: Aaron B. Morton, Columbia, MO (US); Richard K. Brow, Columbia, MO (US); Steven S. Segal, Columbia, MO (US); Julie A. Semon, Columbia, MO (US)

(21) Appl. No.: 18/688,413

(22) PCT Filed: Sep. 1, 2022
(86) PCT No.: PCT/US2022/042374

§ 371 Date: Mar. 1, 2024

Related U.S. Application Data
(60) Provisional application No. 63/260,858, filed on Sep. 2, 2021.

(51) Int. Cl. A61K 33/42; A61K 33/00; A61K 33/08; A61K 33/22; A61K 33/24; A61K 33/30; A61K 33/34; A61P 21/00; C03C 3/19; C03C 4/00

(52) U.S. Cl. CPC A61K 33/42; A61K 33/00; A61K 33/08; A61K 33/22; A61K 33/24; A61K 33/30; A61K 33/34; A61P 21/00; C03C 3/19; C03C 4/0014; C03C 2204/00

(57) ABSTRACT

Compositions and methods for improving the regeneration of soft tissues as a result of injury or disease are provided. Various compositions are disclosed including a bioactive glass composition derived from calcining a reactant composition. Methods for treating injured or diseased skeletal muscle comprising contacting the injured or diseased skeletal muscle with the bioactive glass composition are also disclosed.

### CROSS REFERENCE TO RELATED APPLICATIONS

This application claims priority to U.S. Provisional Patent Application No. 63/260,858 filed Sep. 2, 2021, which is hereby incorporated by reference in its entirety.

### FIELD OF THE INVENTION

Compositions and methods for improving the regeneration of soft tissues as a result of injury or disease are provided. Particularly, bioactive glass compositions for contacting and treating the tissues are described.

### BACKGROUND OF THE INVENTION

Acute trauma is a leading cause of death and disability in the United States. Civilians incur debilitating falls, vehicular crashes and machine injuries while military personnel are subject to combat wounds. Injuries encompassing damage to multiple tissue components are subject to complications including ischemia, denervation and necrosis. Advances in surgical techniques have increased the prevalence of tissue reconstruction, yet, half of affected patients remain severely impaired seven years post operation. Also, soft tissue diseases like muscular dystrophy are subject to repetitive damage as a result of fragile muscle tissue.

In humans, skeletal muscle comprises ˜40% of body mass, facilitates temperature regulation, and generates forces to sustain breathing and locomotion. Due to its location throughout the body, skeletal muscle is prone to impact trauma from motor vehicle accidents, penetration wounds, surgical repair, and overuse injuries. Skeletal muscle possesses a robust regenerative response owing to its population of quiescent muscle stem cells (satellite cells) associated with mature skeletal muscle fibers, residing between the sarcolemma and basement membrane. Following injury, satellite cells activate, proliferate, and differentiate into myoblasts prior to fusing into new myotubes or to the ends of damaged muscle fibers. While skeletal muscle can regenerate, limitations exist. In particular, when injury is too severe, like that of volumetric muscle loss (VML; defined as >20% loss of mass), the muscle does not regenerate and instead results in irreversible scarring, fibrosis, and loss of function. Advances in clinical practice have improved patient outcomes through tissue grafting including autografts, allografts, and xenografts. Even so, limitations arise (immunological rejection and inflammation) with regeneration strategies. In addition to VML, irreversible scarring, fibrosis and loss of function is observed in patients suffering from muscular dystrophy.

For example, Duchenne muscular dystrophy (DMD) is an x-linked, recessive chromosomal mutation of the gene dystrophin, affecting 1 in 5,000 males. In healthy muscle, dystrophin is responsible for maintaining integrity, flexibility, and stability of the muscle fiber membrane (sarcolemma) by anchoring the intracellular F-actin cytoskeleton to the extracellular matrix through the dystrophin-glycoprotein complex. In DMD, the deficiency of dystrophin leads to sarcolemma damage by contractile forces, especially eccentric (lengthening) contractions (e.g., walking down stairs), resulting in increased permeability of myofibers to ions and small molecules. Therapeutic approaches have focused on two strategies: 1) restoring the gene dystrophin (or dystrophin surrogate molecules), or 2) mitigating the secondary consequences caused by dystrophin deficiency. While FDA-approved and pipeline therapies have therapeutic potential, they also are fraught with draw backs that include dismal increases in dystrophin protein (<1% with FDA-approved gene editing drugs, Vyondys and Exondys) with no improvement in muscle function, and secondary consequences of systemic, frontline medications. Mutation therapy has only been approved for 15% of patients. Corticosteroids can only help slow the progression of DMD. Children with DMD exhibit limb muscle dysfunction from repetitive tearing of the muscle as early as 2 years of age, which progresses to immobility by ˜15 years of age due to an inability of a muscle to keep pace with constant injury that results from weakness in myofiber structure. This decline in mobility is the primary burden cited by families of children with DMD. Such debilitating muscle injuries and diseases accelerate the need for biomimetic scaffolds to direct skeletal muscle regeneration.

Accordingly, there is an urgent need for new approaches that improve the regeneration of soft tissues.

### BRIEF SUMMARY OF THE INVENTION

Various compositions are disclosed herein including a bioactive glass composition derived from calcining a reactant composition comprising: about 10 wt. % to about 40 wt. % B₂O₃; about 15 wt. % to about 40 wt. % P₂O₅; about 10 wt. % to about 25 wt. % CaO; about 5 wt. % to about 20 wt. % Na₂O; and optionally about 2 wt. % to about 10 wt. % CoO, about 0.5 wt. % to about 2 wt. % ZnO, about 0.1 wt. % to about 1 wt. % CuO, or a combination thereof.

Various methods are also disclosed herein including a method for treating injured or diseased skeletal muscle comprising contacting the injured or diseased skeletal muscle with an effective amount of any of the bioactive glass compositions as described herein.

The disclosure is further directed to a method for treating injured or diseased brain or nerve tissue comprising contacting the injured or diseased brain or nerve tissue with an effective amount of any of the bioactive glass compositions as described herein.

Other objects and features will be in part apparent and in part pointed out hereinafter.

### BRIEF DESCRIPTION OF THE SEVERAL VIEWS OF THE DRAWING

FIGS. 1A and 1B depict microvessels analyzed following intravital image acquisition. Area of the 2-mm punch injury shown by black circle. Scale bar=1 mm. FIG. 1A depicts microvessels treated with CON (saline vehicle treated/control). FIG. 1B depicts microvessels treated with TRIM (Time Release Ion MATRIX) (CoO).

FIG. 1C depicts the percent of the injury occupied by vessels for saline vehicle treated/control (CON) and Time Release Ion MATRIX (TRIM) (CoO), indicating that regeneration within the sites of injury does not appear to be different between CON and TRIM (CoO) indicated by the larger average bar.

FIGS. 1D and 1E depict confocal image stacks illustrating fluorescent vessels in the gluteus maximus muscle (GM) at 21 dpi. Images were acquired from the center of the injury. Scale bars=400 μm. FIG. 1D depicts treatment with CON. FIG. 1E depicts treatment with TRIM (CoO).

FIG. 1F depicts a quantification of the percent of vessels in the injury for CON and TRIM (CoO), revealing the average microcirculation density at the site of injury (percent of vessels in the injury) of CON to be higher than TRIM.

FIGS. 2A and 2B depict confocal image stacks that illustrate myofibers in GM muscle at 21 dpi, acquired from the center of the injury (see FIG. 1A-F). Myofibers in GM receiving saline (CON) were sparse and disorganized. Large gaps (black) were void of muscle fibers within the injury site. Bar=400 μm. FIG. 2A depicts treatment with CON. FIG. 2B depicts treatment with TRIM (CoO).

FIG. 2C depicts the percent of myofibers regenerated with CON and TRIM (CoO) treatment, revealing that TRIM (CoO) treated mice had increased myofiber density average than CON.

FIGS. 2D and 2E depict laminin borders and myofiber nuclei depicting cross sections of the GM. The cross section contains healthy muscle at the top and bottom with the injury in the middle (dotted circle). Scale bars=200 μm. FIG. 2D depicts treatment with CON. FIG. 2E depicts treatment with TRIM (CoO).

FIG. 2F depicts muscle recovery in cross-sections after CON and TRIM treatment, revealing that TRIM's average muscle thickness approaches 1 while CON's average muscle thickness averaged approximately 0.75.

FIG. 3A depicts representative images of mouse GM cross sections. Following injury, muscles were treated with sham saline(S) or with TRIMCuZn (BPCuZn) at 3 days post injury (dpi) and studied at 8 dpi. DAPI staining shows central nuclei of regenerated myofibers, laminin labels cell borders (top). Laminin and eMyHC costaining is also shown (bottom). Scale bar=100 pm and applies to all images.

FIG. 3B depicts percent of eMyHC positive fibers for sham saline treatment(S) at 8 dpi and TRIMCuZn (BPCuZn) treatment at 0 and 8 dpi. Loss of embryonic myosin heavy chain (eMyHC) indicates TRIMCuZn accelerates myofiber maturation and improves angiogenesis. n=2-3/group, data reported as mean with SEM.

FIG. 3C depicts percent of regenerated fibers for sham saline treatment(S) at 8 dpi and TRIMCuZn (BPCuZn) treatment at 0 and 8 dpi.

FIG. 4A depicts a representative image and wet weights of TAs in 7 mo old DBA mice untreated or following 14 days after Dystrophix (CoO) injection. The TA treated with Dystrophix had greater mass than the 3 untreated TAs.

FIG. 4B depicts a representative image and weights of wet EDL in 7 mo old DBA mice untreated or following 14 days after Dystrophix (CoO) injection. The EDL treated with Dystrophix has greater mass than the 3 untreated.

FIG. 4C depicts representative images of TA cross sections from DBA mice untreated and treated with Dystrophix. Embryonic myosin heavy chain (eMyHC) is less in Dystrophix-treated muscle, which also has more central nuclei compared to untreated. Myofiber borders labeled with laminin. Scale bar=250 μm.

FIG. 5 depicts percent of peak force following injury after treatment with saline, BP, or Dystrophix (CoO). Maximum tetanic contractions were recorded 14 days after treating the left TA of mdx mice with either saline, BP, or Dystrophix (CoO). Following a brief rest, the TA underwent three lengthening contractions with rest between. Maximum tetanic contractions were recorded again to determine what percentage of the initial maximum tetanic force could be elicited following eccentric injury.

FIG. 6A depicts representative images of fibers with CD31 staining for no treatment 0 dpi, BPCuZn 0 dpi, 8 dpi, and BPCuZn 8 dpi samples. Scale bar=200 μm.

FIG. 6B depicts the microvessel area/fiber as a percent of control for no treatment 0 dpi, BPCuZn 0 dpi, 8 dpi, and BPCuZn 8 dpi samples. n=2-3/group.

FIG. 7A depicts representative samples of fibers treated with saline and dystrophix stained from laminin, MyHC, and DAPI.

FIG. 7B relative frequency for myofiber cross section area for saline and dystrophix treated mice.

Corresponding reference characters indicate corresponding parts throughout the drawings.

### DETAILED DESCRIPTION OF THE INVENTION

A strategy was developed for in vivo regeneration using inorganic biocompatible ceramics (biocompatible glass) in the form of powders suspended in inert solutions (e.g., sterile 0.9% saline) prior to injection into the site of injury in order to enhance local tissue scaffolding and repair response. The composition candidates of biocompatible glass have shown similar beneficial effects on the structure and function of skeletal muscle in healthy mice injured with a punch biopsy as well as diseased dystrophic mice. Such enhancement of muscle regeneration and dystrophic muscle structure and function may rely primarily upon the borophosphate particles but also on other additives [e.g., CoO (may enhance hypoxia inducible factor 1a), CuO (may be angiogenic), ZnO (may be anti-inflammatory)]. Previous experiments have demonstrated that both borate- and phosphate-based glasses exert adhesion and structural support of bone and tooth enamel through the formation of calcium phosphate layers on the surface of the glass. The biocompatible glass of the instant invention is created by combining borate and phosphate at ratios that slow the rate of dissolution at neutral pH, without affecting the local pH. It is thought that it forms a calcium phosphate layer that serves as a “biomimetic micro scaffold” for damaged and diseased myofibers. This effect can localize to the extracellular glycoprotein portion of the dystrophin-glycoprotein complex to stabilize myofiber structure in place of dystrophin. When injected locally into a myofascial compartment, it appears to affect all muscles within the compartment and can thereby serve as a therapy for preserving myofiber integrity and physical mobility in patients with muscle injury or muscular dystrophy.

Time Release Ion Matrix (TRIM) is a borate phosphate based amorphous non-crystalline solid (bioactive glass) containing cobalt ions. When ground into a powder, suspended in solution, and injected into damaged soft tissue (from trauma or disease), the material appears to significantly increase the rate of soft tissue regeneration. Other bioactive glass compositions have been used to treat volumetric muscle loss (e.g., borate aluminate glass powder); however, no other compositions have been shown to stimulate the regeneration of injured skeletal muscle, dystrophic muscle, blood vessels or peripheral nerves. Reported herein is that injured, normal (free of disease) skeletal muscle as well as dystrophic skeletal muscle can improve in both size and quality following TRI Matrix treatment. Additional applications may include brain, and peripheral nerves for regeneration of soft tissue via hypoxia mimetic pathways.

Various compositions are disclosed herein including a bioactive glass composition derived from calcining a reactant composition comprising: about 10 wt. % to about 40 wt. % B₂O₃; about 15 wt. % to about 40 wt. % P₂O₅; about 10 wt. % to about 25 wt. % CaO; about 5 wt. % to about 20 wt. % Na₂O; and optionally about 2 wt. % to about 10 wt. % CoO, about 0.5 wt. % to about 2 wt. % ZnO, about 0.1 wt. % to about 1 wt. % CuO, or a combination thereof.

The reactant composition can comprise: about 10 wt. % to about 40 wt. % B₂O₃; about 15 wt. % to about 40 wt. % P₂O₅; about 10 wt. % to about 25 wt. % CaO; about 5 wt. % to about 20 wt. % Na₂O; and about 2 wt. % to about 10 wt. % CoO.

The reactant composition can comprise: about 33 wt. % to about 37 wt. % B₂O₃; about 33 wt. % to about 37 wt. % P₂O₅; about 13 wt. % to about 18 wt. % CaO; about 11 wt. % to about 14 wt. % Na₂O; and about 3 wt. % to about 5 wt. % CoO.

The reactant composition can comprise: about 30 wt. % to about 40 wt. % B2O3; about 20 wt. % to about 40 wt. % P₂O₅; about 10 wt. % to about 20 wt. % CaO; about 11 wt. % to about 18 wt. % Na₂O; and about 3 wt. % to about 10 wt. % CoO.

The reactant composition can comprise: about 30 wt. % to about 40 wt. % B₂O₃; about 30 wt. % to about 40 wt. % P₂O₅; about 10 wt. % to about 20 wt. % CaO; about 10 wt. % to about 15 wt. % Na₂O; about 0.5 wt. % to about 2 wt. % ZnO; and about 0.1 wt. % to about 1 wt. % CuO.

The reactant composition can comprise: about 33 wt. % to about 37 wt. % B₂O₃; about 33 wt. % to about 37 wt. % P₂O₅; about 13 wt. % to about 18 wt. % CaO; about 11 wt. % to about 14 wt. % Na₂O; about 0.8 wt. % to about 1.2 wt. % ZnO; and about 0.3 wt. % to about 0.5 wt. % CuO.

The reactant composition can comprise: about 33 wt. % to about 37 wt. % B₂O₃; about 33 wt. % to about 37 wt. % P₂O₅; about 13 wt. % to about 18 wt. % CaO; and about 11 wt. % to about 14 wt. % Na₂O;

The calcining can be performed by heating the reactant composition at a temperature below the melting temperature of the reactant composition. The temperature for calcining can be from about 800° C. to about 1300° C. or from about 1000° C. to about 1150° C. The reactant composition can further comprise phosphoric acid.

The bioactive glass composition can be used to form calcium phosphate. The bioactive glass composition can maintain a neutral pH as it degrades, which encourages the formation of tri-calcium phosphates. This is in contrast to other bioactive glasses that create an alkaline pH environment, which encourages the formation of hydroxyapatites.

The disclosure is further directed to a method for treating injured or diseased skeletal muscle comprising contacting the injured or diseased skeletal muscle with an effective amount of any of the bioactive glass compositions as disclosed herein. The injured or diseased skeletal muscle can have an increase in average myofiber area after at least 8 days of treatment with the bioactive glass composition as compared to an injured or diseased skeletal muscle that undergoes an otherwise similar treatment with saline. The injured or diseased skeletal muscle can have a lower embryonic myosin heavy chain (eMyHC) concentration after at least 5 days of treatment with the bioactive glass composition as compared to an injured or diseased skeletal muscle that undergoes an otherwise similar treatment with saline. The injured or diseased skeletal muscle can have an increased muscle mass after at least 10 days of treatment with the bioactive glass composition as compared to an injured or diseased skeletal muscle that undergoes an otherwise similar treatment with saline. The injured or diseased skeletal muscle can have an increased muscle peak force after at least 10 days of treatment with the bioactive glass composition as compared to an injured or diseased skeletal muscle that undergoes an otherwise similar treatment with saline. The injured or diseased skeletal muscle can have an increased angiogenesis after at least 5 days of treatment with the bioactive glass composition as compared to an injured or diseased skeletal muscle that undergoes an otherwise similar treatment with saline.

The injured or diseased skeletal muscle can be injured skeletal muscle. The injured skeletal muscle can be a pulled muscle, traumatically injured muscle, ruptured muscle, injured muscle resulting from muscle overuse or misuse, or a combination thereof. The injured muscle resulting from muscle overuse or misuse can be the result of a sports injury.

The injured or diseased skeletal muscle can be a diseased skeletal muscle. The diseased skeletal muscle can be dystrophic skeletal muscle, cachexic skeletal muscle, sarcopenic skeletal muscle, or a combination thereof.

The disclosure is further directed to a method for treating injured or diseased brain or nerve tissue comprising contacting the injured or diseased brain or nerve tissue with an effective amount of the any of the bioactive glass compositions as disclosed herein.

For any of the methods disclosed herein, the bioactive glass composition can maintain a neutral pH as it degrades.

Having described the invention in detail, it will be apparent that modifications and variations are possible without departing from the scope of the invention defined in the appended claims.

### EXAMPLES

The following non-limiting examples are provided to further illustrate the present invention.

### Summary

Skeletal muscle is vulnerable to trauma from motor vehicle accidents, penetration wounds, surgical repair, and overuse injuries. While skeletal muscle can regenerate, limitations exist. In particular, when the injury is too severe, like that of volumetric muscle loss (VML; defined as >20% loss of mass), the muscle does not regenerate and instead results in irreversible scarring, fibrosis, and loss of function. In addition, Duchenne's Muscular Dystrophy (DMD) results in depletion of a muscle's regenerative capacity due to repetitive myofiber tearing. Biomaterials have shown promise enhancing muscle regeneration following VML.

Experiments were performed to test that the biomaterial timed-release ion matrix (TRIM) enhances microvascular and myofiber regeneration in skeletal muscle following chemical injury, VML, and in conditions of DMD. To induce chemical injury BaCl₂ was used while a biopsy punch (2-mm diameter) was used to induce VML, both in the left gluteus maximus muscle (GM) of female C57BI/6 and Cdh5-mTmG mice (age, 4-5 months). Dystrophic mice [n=2; mdx^{+/+} (3-5 months old)] were used for eccentric injury experiments following single injections of TRIM or vehicle into the left tibialis anterior muscle (TA), while DBA^{mdx} mice [n=1; (7 months old)] were used as a model of sever dystrophy to determine if TRIM could restore TA muscle mass. Mice were divided into two groups: saline vehicle treated (CON) or TRIM treated. For treated mice, 250 μg of TRIM powder was suspended in 0.9% sterile saline (5 μg/pL) and 70 μL of TRIM solution was injected beneath the GM at 3 days post injury (dpi) for BaCl₂ injuries, 7 dpi for VML injuries, and 50 μL injected into TAs of dystrophic mice 10 days prior to data collection. For CON mice, 70 μL of 0.9% saline solution without TRIM was injected at 7 dpi in the GM as a negative control, while 50 μL was injected into the TAs of dystrophic mice. Muscles were evaluated by intravital microscopy, confocal microscopy, and tissue sections. Intravital microscopy did not reveal differences between TRIM or CON regarding the area within the injury occupied by blood vessels while confocal z-stacks suggest that TRIM reduced vascular density within the injured area. In contrast, both confocal z-stacks and muscle cross sections suggest TRIM enhanced myofiber regeneration in all mice treated as well as enhanced dystrophic muscle resistance to injury. The findings suggest that TRIM treatment may be beneficial for muscle fiber regeneration following chemical injury of skeletal muscle, VML injury, and in conditions of muscular dystrophy.

### Example 1: Materials and Methods

The following materials and used were used throughout the rest of the Examples.

### Animals

Mice were selected because skeletal muscle structure and function are conserved across species and the invasive nature of these experiments prevented experimentation in humans. All experiments were approved by the Animal Care and Use Committee at the University of Missouri. Male C57BI/6 mice (n=3; ˜4 months of age) were selected for chemical injury and TRIM treatment. Female Cdh5-mTmG (endothelial cell green fluorescent protein (GFP) reporter; ˜4 months of age) were used to visualize microvascular regeneration after volumetric muscle loss injury (VML). The endothelial specific cre recombinase Cdh5 Cre-ERT2 was selected (Wang, Y., et al. (2010). Nature, 465(7297)) and crossed with an mTmG reporter to create a validated endothelial cell (EC) reporter mouse (Muzumdar, D., et al. (2007). genesis, 45(9), 593-605.). Mice were divided into two groups: saline vehicle treated (CON), n=3 or biocompatible ceramic [Timed-release ion matrix (TRIM)], treated, n=2. Mice were maintained under a 12:12 hr light/dark cycle and housed with bedding cubes and free access to food and water.

Two strains of dystrophic mice were used to evaluate the effects of TRIM upon dystrophic muscle. C57BI/6 mdx+/+ mice (n=2; ˜4 months of age) were used to evaluate the effect of TRIM on dystrophic muscle's resistance to injury while D2.B10 Dmdmdx mice (n=1; 7 months of age) present with a more severe muscle phenotype and were used to determine TRIM's ability to restore dystrophic muscle quality and mass.

### Tamoxifen Injections

To induce Cre-ERT2 recombination and eGFP expression in the endothelium, mice were restrained by trained personnel and 100 μL of tamoxifen solution (1 mg tamoxifen+5% ethanol in corn oil) was injected intraperitoneal with a 27-gauge on three consecutive days as reported (Biomimetic Bioactive Biomaterials: The Next Generation of Implantable Devices. (2017). ACS Biomaterials Science & Engineering, 3(7), 1172-1174.). All mice were studied 7 days after the initial tamoxifen injection.

### Generation of the Time Release Ion Matrix (TRIM)

TRIM is generated by mixing the dry, powdered components and placing them in a platinum crucible.

For these experiments, three different compositions of TRIM were used:

Candidate material composition of biocompatible glass particles (values are % by weight):

CoO: 34.6% B₂O₃, 35.3% P₂O₅, 14.0% CaO, 12.3% Na₂O, 3.8% CoO

BPCuZn: 35% B₂O₃, 35.6% P₂O₅, 16.2% CaO, 11.8% Na₂O, 1% ZnO, 0.4% CuO

BP: 34.9% B₂O₃, 35.8% P₂O₅, 16.9% CaO, 12.4% Na₂O

If phosphoric acid was required, it was then slowly stirred into the dry components. The batch was calcined overnight to evolve water prior to melting (1000-1150° C.) for 60 minutes, then stirred with a platinum rod for 30 minutes. The melted TRIM mixtures were ground to form particles <20 μm using a Spex mill. A solution of the TRIM particles is created (5 mg/mL in 0.9% sterile saline) and injected as described below.

### BaCl₂ for Muscle Injury and TRIM Treatment

To induce chemical-induced muscle injury in vivo, mice were anesthetized with ketamine and xylazine (100 mg/kg and 10 mg/kg respectively; intraperitoneal injection), the skin was shaved over the muscle of interest, then 1.2% BaCl₂ was injected unilaterally into the TA [50 μL; (Hench, L. L., & Thompson, I. (2010). Journal of The Royal Society Interface, 7(suppl_4), S379-S391.)] or under the GM [75 μL; (Hench, L. L., & Polak, J. M. (2002). Science, 295(5557), 1014)] as described. Mice were kept warm during recovery and then returned to their cage.

### Punch Biopsy Injury for VML and TRIM Treatment

A mouse was anesthetized with ketamine/xylazine (100 mg/kg and 10 mg/kg, respectively) IP and rested on an aluminum warming plate to maintain body temperature at 37° C. If needed, supplemental injections (˜20% of the initial) were given to maintain a stable state of anesthesia confirmed by lack of withdrawal to a tail or toe pinch every 15 minutes. Skin covering the left GM was shaved and sterilized by swabbing with Betadine (10% povidone-iodine topical solution) and then 3 times with alcohol wipes. While viewing through a stereomicroscope, the mouse was positioned on its abdomen and a ˜5 mm incision was made through the overlying skin to gain access to the GM. The exposed tissue was continuously irrigated with physiological salt solution (PSS). Care was taken to avoid injuring the blood vessels of the GM. Using a sterile 2 mm diameter punch biopsy (Anderson, S., et al. (2019). Tissue Engineering Part C: Methods, 25(2), 59-70), a local injury was made in the GM. 2 mm was chosen to represent a model of VML that is less than the critical threshold of muscle loss for non-regeneration (Anderson, S., et al. (2019). Tissue Engineering Part C: Methods, 25 (2), 59-70). In order to keep the locations of the punch injury consistent, a custom measuring device of 1 cm by length, 0.5 cm by width was placed along the lumbar spine to provide a reference point in the GM. For VML mice treated TRIM, 250 μg of powder was suspended in 0.9% sterile saline prior to injecting beneath the GM. For CON mice, 70 μL of 0.9% saline solution was injected under the muscle at 7 days post injury (dpi) to mimic the treatment. The skin incision was closed with 4 to 5 discontinuous stitches placed through the skin using sterile 6-0 nylon suture. For dystrophic mice, 250 μg of powder was suspended in 0.9% sterile saline prior to injecting into the left tibialis anterior (TA) muscle while mice were under anesthesia to prevent moving. Mice were kept warm and monitored until ambulation was restored (2-3 hours), then returned to their cage and observed daily. Following data collection at 21 dpi for VML and 14 days for dystrophic mice, a mouse was killed by cervical dislocation under anesthesia.

### Intravital Microscopy of the GM

Mice were anesthetized in order to prepare the GM for intravital microscopy (in vivo imaging of the microcirculation) while preserving the integrity of its vascular supply as described (Fernando, C. A., et al. (2019). The Journal of Physiology, 597(5), 1401-1417.). Ketamine/xylazine was injected IP and the skin overlying the GM was shaved to remove hair. The mouse was transferred to a warming plate at a temperature of 37° C. to maintain body temperature. Through a stereomicroscope, an incision along the spinal cord was made in the overlying skin. Excess connective tissue and fat were removed using microdissection while avoiding major blood vessels. The exposed GM was continuously irrigated with PSS. The GM was then dissected free from its origin along the lumbar fascia, sacrum, and iliac crest and reflected away from the body to expose its vascular supply. It was then spread onto the surface of a transparent rubber pedestal and pinned down at the edges approximating in situ dimensions. Spreading and securing the tissue over the pedestal produced a thin flat preparation suitable for high resolution imaging of the microvasculature. Any other exposed tissues were covered with Saran wrap to prevent dehydration during intravital imaging.

After the completion of surgery, the mouse preparation was transferred to the stage of a Nikon 600fn intravital microscope and continuously irrigated with PSS equilibrated with 5% CO2/95% N2. Digital images were acquired in Piper Software with a low light CMOS FP-Lucy camera (Stanford Photonics) and Long Working Distance (LWD) 4× and 10× objectives (Nikon) to image the entire punch injury. Following intravital microscopy, the GM was dissected and trimmed to include the area containing the punch injury for confocal imaging and subsequent freezing to section for histology.

### Muscle Force Measurements of TA and Mass of TA and EDL

The TA was prepared for in situ measurements as described (Wang, Y., et al. (2010). Nature, 465(7297), 483-486). Briefly, in an anesthetized mouse, a 2-0 suture was placed around the left patellar tendon. The sciatic nerve was isolated and severed proximal to the TA for stimulation of muscle force through electrode via a Grass™ stimulator. The distal tendon of the TA was isolated, secured in 2-0 suture, then severed from its insertion. The mouse was placed prone on a plexiglass board and the patellar tendon was secured to a vertical metal peg immobilized in the board. The distal TA tendon was tied to a load beam (LCL-113G; Omega, Stamford, CT, USA) coupled to a Transbridge amplifier (TBM-4; World Precision Instruments, Sarasota, FL, USA). The load beam was attached to a micrometer for adjusting optimal length (Lo) as determined during twitch contractions at 1 Hz (Hench, L. L., & Polak, J. M. (2002). Science, 295(5557), 1014). A strip of KimWipe® was wrapped around the TA and physiological salt solution irrigated the TA (3 mL min−1) and maximum force was evaluated for at 120 Hz with Power Lab acquisition software (ADInstruments, Colorado Springs, CO, USA) before and after eccentric contractile injury. After establishing optimal muscle length and performing three warm-up contractions, a maximal tetanic contraction was evaluated, then the muscle was lengthened ˜40% during three maximal contraction conditions as described (Muzumdar, D., et al. (2007). genesis, 45(9), 593-605). Following a two-minute rest, maximal tetanic contractions were obtained again to determine the percentage of force lost following eccentric injury. Following data collection, muscle length was measured and both TA and EDL muscles were removed to evaluate mass.

### Histochemistry (Imaging and Analysis)

The GM specimen was transferred to the stage of a laser scanning confocal microscope to image microvessels and myofibers. Following confocal image acquisition, optimal cutting temperature (OCT) compound was poured into a shallow cryomold and the dissected GM was oriented in the center lying flat. A 2-mm length of silk suture was placed next to the GM in the cryomold to indicate the location of the VML injury and was frozen in isopentane cooled in liquid nitrogen. The frozen GM was wrapped in foil, labelled for reference, and stored at −80° C. until processed for sections.

### Staining of Frozen Sections

Frozen GM and TA sections were cut at a thickness of 10 μm with a cryostat (HM 550 Cryostat, Thermo Scientific, Waltham, MA) and stained, as described (Morton, A. B., et al. (2019). Redox Biology, 20, 402-413) for laminin (Thermo Scientific #RB-9024-R7), myosin heavy chain and embryonic myosin (Hybridoma Bank A4.840 s IgM 1:15), and mounted with Prolong Gold containing DAPI (Thermo Fisher).

### ImageJ Analysis

For the intravital images, vessels were analyzed to evaluate the amount of microvascular regeneration. For the confocal images, both vessels and myofibers were analyzed separately to quantify each component. Images of muscle cross-sections were acquired to evaluate muscle thickness as described below.

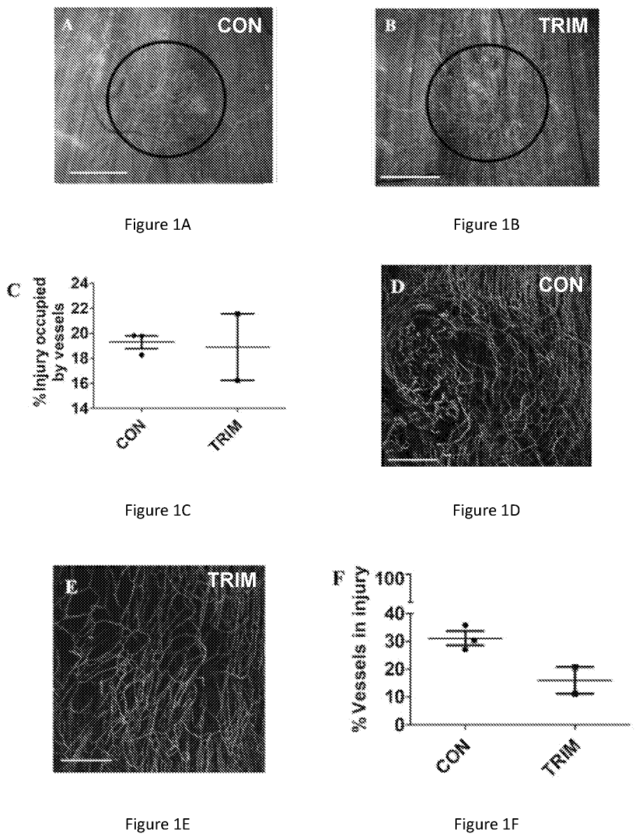

Images acquired with the Nikon 600fn intravital microscope were analyzed with open access software ImageJ and Microsoft PowerPoint. A reference image of the punch biopsy injury was used to confirm the original size when analyzing both CON and TRIM treated GM. Using PowerPoint, the image of the specimen was overlayed on top of the reference image to define the area of the injury. These images were combined and saved as one TIFF file with a circle from PowerPoint tools outlining the area of injury (FIG. 1, A and B). The combined images were converted to 32-bit grayscale after being imported into ImageJ. To identify vessels within the area of injury (A01), background threshold was established, images were converted to binary where the background (non-vasculature) was white, and tissue component of interest (vessel) was black. These images were used to determine the extent of myofiber regeneration and revascularization, defined as the percentage of total A01 occupied by microvessels.

Confocal images were acquired with a 10× objective at ×0.75 digital zoom on an inverted laser scanning confocal microscope (TCS SP8, Leica Microsystems Buffalo Grove, IL, USA) using Leica LAX software. Image stacks (thickness, ˜70 pm) were used to resolve VML morphology (Morton, A. B., et al. (2019). Skeletal Muscle, 9(1), 27) using ImageJ software (NIH, open access). Confocal Z-stacks acquired in two color channels were separated into GFP (ECs) and TD tomato (myofibers) following import into ImageJ. Each color channel image was converted to 32-bit grayscale using the threshold guidelines described above. Area occupied in black (vessels or myofibers) was expressed as percentage of the total A01. Vessel and muscle Images were analyzed separately.

For confocal image analyses, % vessel area and % muscle area were compared between treatments and across time points. The experimenter was blinded to the experimental group for both analyses. The coefficient of variation was <5% for the data collected.

To evaluate muscle thickness, muscle cross sections were analyzed at 5 different locations: at the section ends (thickest, uninjured segments), section center, and injured area midway between the section center and section ends. The thickness of uninjured section ends were averaged (UT) followed by the thickness of injured sections (IT). Final values were calculated using the following equation: IT/UT=Muscle Recovery. A ratio of 1 indicated recovery of muscle thickness throughout the injured area, while a ratio of <1 indicated less regeneration.

### Example 2: TRIM Does Not Appear to Enhance Vascular Density in GM Following VML

Following muscle injury in the gluteus maximus (GM) with a 2 mm biopsy punch, the microcirculation and muscle within the injury site was removed. To nourish and maintain myofibers, the microcirculation must regenerate following injury. Intravital imaging depicts endothelial cell green fluorescent protein (GFP) (FIG. 1, A and B) at 21 days post injury (dpi). The addition of TRIM at 7 dpi did not appear to alter vascular density in the injured area by day 21 as evaluated by fluorescent image analysis (CON=19.5% of the A01; TRIM treated=19%; FIG. 1C).

Greater detail of vascular density at 21 dpi was resolved with confocal microscopy images acquired from the center of the VML injured site (FIGS. 1D and 1E). The vascular density in the TRIM-treated (15% of A01) GM appeared less than CON (30.2%) (FIG. 1F).

### Example 3: TRIM Enhances Myofiber Regeneration Following VML

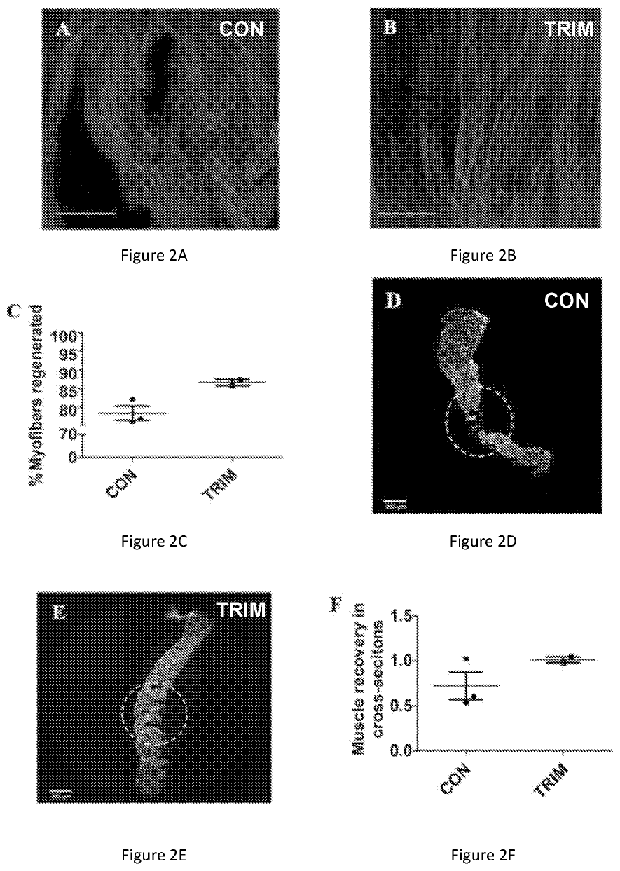

Confocal images depict myofiber regeneration following injury (FIGS. 2A and 2B) at 21 dpi. The addition of TRIM appeared to enhance myofiber regeneration in the injured area. Myofibers in GM treated with TRIM (FIG. 2B) were organized in a parallel fashion and more evenly distributed through the injury site when compared to saline-treated controls (CON). Myofiber density appeared to increase with the TRIM (87%) vs. CON (CON=78%) of the A01 (FIG. 2C).

Frozen GM sections were sliced and stained for laminin and myosin heavy chains. Muscle cross-section image analyses revealed TRIM may enhance myofiber regeneration as measured by muscle thickness (FIGS. 2D and 2E). The saline-treated GM (FIG. 2, D) had reduced thickness in the center of the injury. In contrast, TRIM-treated GM (FIG. 2E) had similar thickness throughout both the uninjured and injured muscle. Thus, at 21 days of recovery, the average muscle thickness was greater in TRIM vs. CON. Expressed as the ratio of IT/UT, CON GM averaged in 0.75 while TRIM averaged 1.0 (FIG. 2F).

### Example 4: TRIM Enhances Myofiber Regeneration Following Chemical Injury

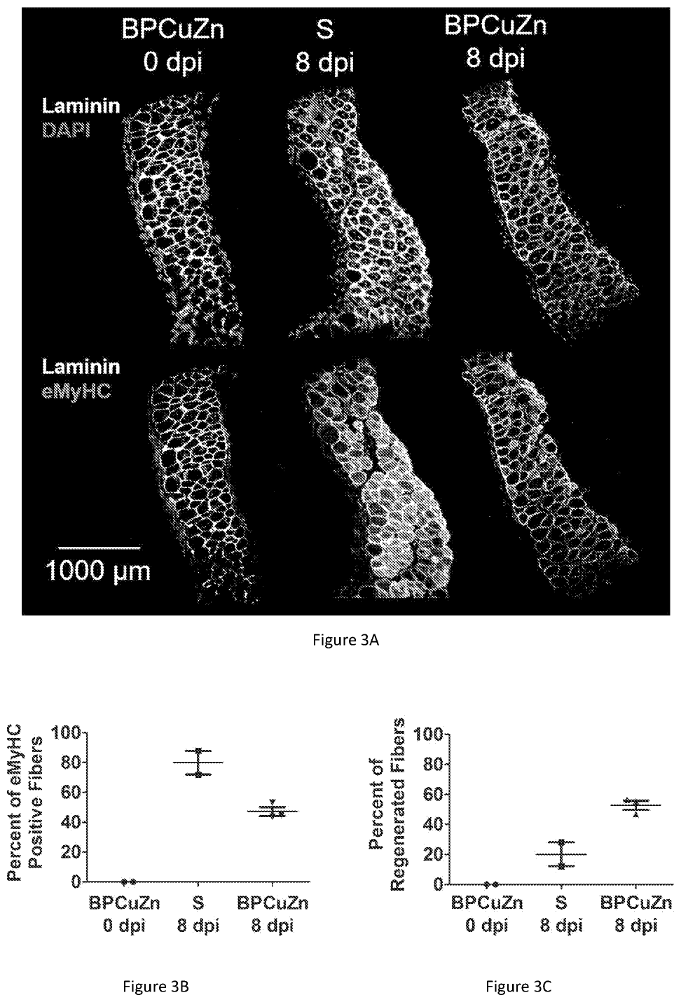

Uninjured BPCuZn treated fibers, saline treated fibers 8 dpi, and BPCuZn treated fibers 8 dpi were stained for eMyHC, laminin, and DAPI (FIG. 3A). The BPCuZn treated 8 dpi fibers had a lower percent of eMyHC positive fibers compared to the saline treated 8 dpi fibers (FIG. 3B). The BPCuZn treated 8 dpi fibers also had a higher percent of regenerated fibers compared to the saline treated 8 dpi fibers and uninjured BPCuZn treated fibers (FIG. 3C).

### Example 5: TRIM Enhances Mass of Severely Dystrophic Muscle

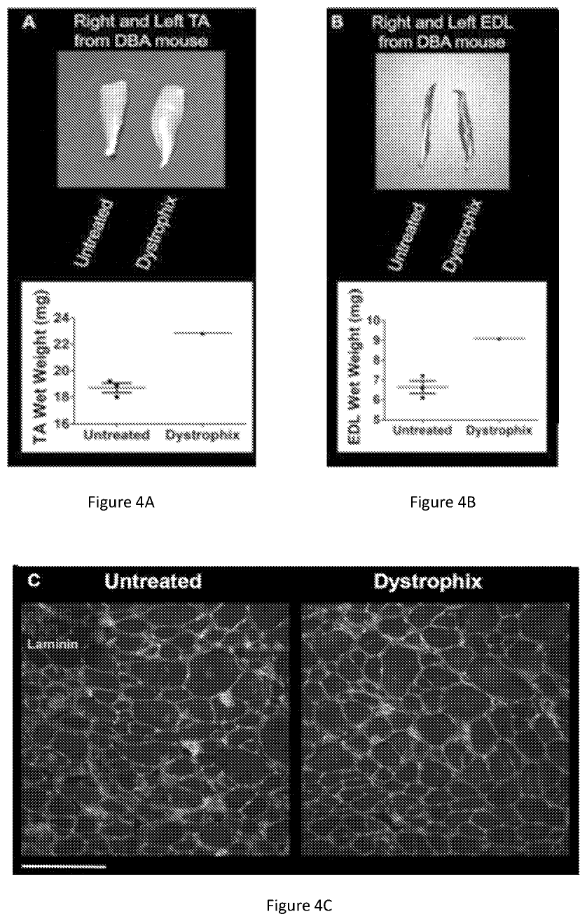

Following a single injection of Dystrophix, muscle mass was increased by ˜20-30% compared to untreated TA and EDL, demonstrating that the muscles of the anterior myofascial compartment in the lower leg respond to a single injection 14 days later (FIG. 4A-4C). Also, only the left limb was treated, therefore, TA and EDL from the contralateral right limb was also harvested to demonstrate that Dystrophix does not possess systemic affects (FIG. 4A-4C, TA and EDL treated with Dystrophix shown with contralateral, untreated limb). To determine if the increase in mass was from myofiber regeneration, muscle cross sections were acquired in 10 μm thick sections and labeled with laminin to identify myofiber borders, with embryonic myosin heavy chain (eMyHC) as a marker of regenerating myofibers, and with DAPI to visualize nuclei. Dystrophix-treated samples presented more centrally located nuclei (a marker of regenerated myofibers) with less fibrosis and eMyHC compared to untreated samples (FIG. 4) indicating augmentation of effective muscle regeneration with more mature myofibers.

Example 6: TRIM Enhances Structure of Dystrophic Muscle

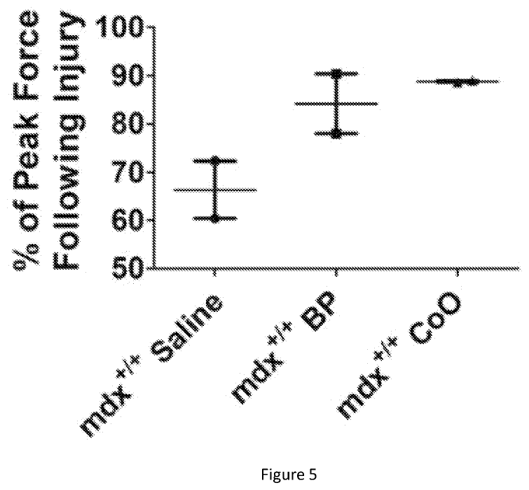

The TAs of Dystrophic mice receiving the single Dystrophix injection demonstrated greater force (i.e., were more resistant to eccentric muscle injury) than saline controls (FIG. 5). When muscle forces produced following eccentric contraction-induced injury were compared to muscle forces produced before injury, Dystrophix treated dystrophic muscle retained ˜90% of preinjury values compared to saline controls, which retained ˜65% their preinjury value.

### Example 7: TRIM Enhances Vascular Growth

0 dpi (days post injury) mice had no treatment. 8 dpi mice were injected with BaCl2 to induce chemical injury and analyzed at 8 dpi. BPCuZn 0 dpi mice were injected with 10 μg BpCuZn/g of body mass and analyzed at 3 dpi. BPCuZn 8 dpi mice were injected with BaCl2 to induce chemical injury. They were then injected with 10 μg BpCuZn/g of body mass at 3 dpi and analyzed at 8 dpi.

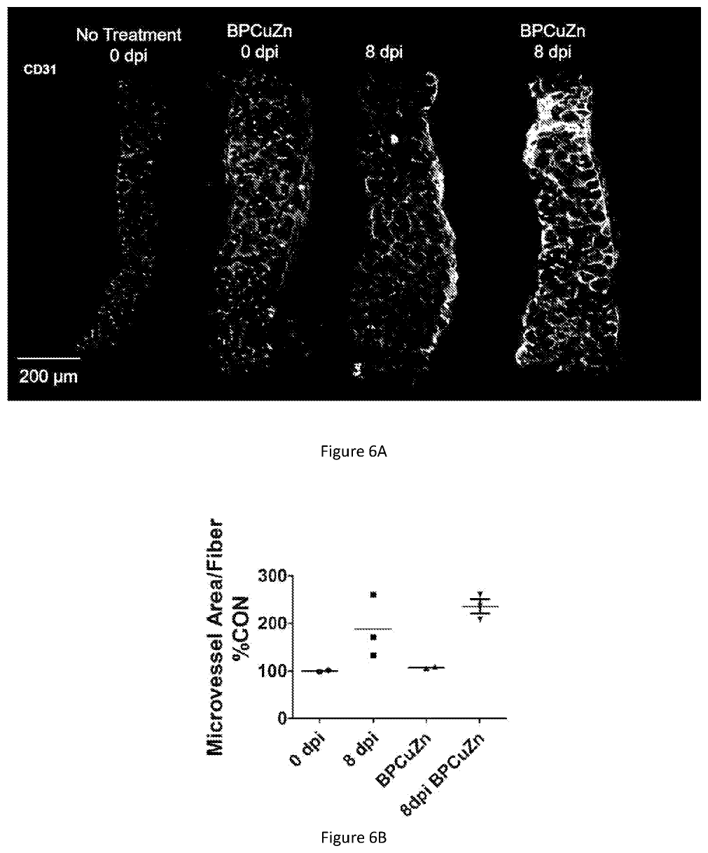

CD31 staining indicates vascular differentiation (FIG. 6A). The relative amount of microvessel area/fiber is slightly higher in 8 dpi BPCuZn mice compared to 8 dpi mice (FIG. 6B). These results suggest that BPCuZn enhances vascular growth.

### Example 8: TRIM Increases Fiber Size

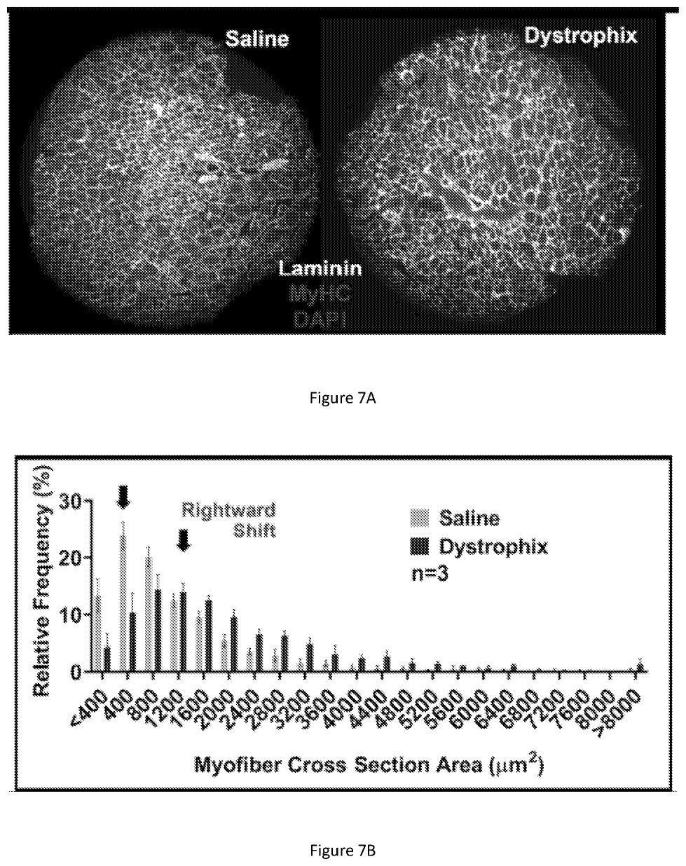

Fibers from saline and Dystrophix treated mice were stained for laminin, MyHC, and DAPI (FIG. 7A). In the quantification of the frequency of myofiber cross section area, treatment with Dystrophix causes a rightward shift compared to the saline control (FIG. 7B), indicating that Dystrophix treatment increases fiber size.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

1. A bioactive glass composition derived from calcining a reactant composition comprising
about 10 wt. % to about 40 wt. % B₂O₃;
about 15 wt. % to about 40 wt. % P₂O₅;
about 10 wt. % to about 25 wt. % CaO;
about 5 wt. % to about 20 wt. % Na₂O; and
optionally about 2 wt. % to about 10 wt. % CoO, about 0.5 wt. % to about 2 wt. % ZnO, about 0.1 wt. % to about 1 wt. % CuO, or a combination thereof.

2. The bioactive glass composition of claim 1, wherein the reactant composition comprises
about 10 wt. % to about 40 wt. % B₂O₃;
about 15 wt. % to about 40 wt. % P₂O₅;
about 10 wt. % to about 25 wt. % CaO;
about 5 wt. % to about 20 wt. % Na₂O; and
about 2 wt. % to about 10 wt. % CoO.

3. The bioactive glass composition of claim 1, wherein the reactant composition comprises
about 33 wt. % to about 37 wt. % B₂O₃;
about 33 wt. % to about 37 wt. % P₂O₅;
about 13 wt. % to about 18 wt. % CaO;
about 11 wt. % to about 14 wt. % Na₂O; and
about 3 wt. % to about 5 wt. % CoO.

4. The bioactive glass composition of claim 1, wherein the reactant composition comprises
about 30 wt. % to about 40 wt. % B₂O₃;
about 20 wt. % to about 40 wt. % P₂O₅;
about 10 wt. % to about 20 wt. % CaO;
about 11 wt. % to about 18 wt. % Na₂O; and
about 3 wt. % to about 10 wt. % CoO.

5. The bioactive glass composition of claim 1, wherein the reactant composition comprises
about 30 wt. % to about 40 wt. % B₂O₃;
about 30 wt. % to about 40 wt. % P₂O₅;
about 10 wt. % to about 20 wt. % CaO;
about 10 wt. % to about 15 wt. % Na₂O;
about 0.5 wt. % to about 2 wt. % ZnO; and
about 0.1 wt. % to about 1 wt. % CuO.

6. The bioactive glass composition of claim 1, wherein the reactant composition comprises
about 33 wt. % to about 37 wt. % B₂O₃;
about 33 wt. % to about 37 wt. % P₂O₅;
about 13 wt. % to about 18 wt. % CaO;
about 11 wt. % to about 14 wt. % Na₂O;
about 0.8 wt. % to about 1.2 wt. % ZnO; and
about 0.3 wt. % to about 0.5 wt. % CuO.

7. The bioactive glass composition of claim 1, wherein the reactant composition comprises
about 33 wt. % to about 37 wt. % B₂O₃;
about 33 wt. % to about 37 wt. % P₂O₅;
about 13 wt. % to about 18 wt. % CaO; and
about 11 wt. % to about 14 wt. % Na₂O,

8. The bioactive glass composition of claim 1, wherein the calcining was performed by heating the reactant composition at a temperature below the melting temperature of the reactant composition.

9. The bioactive glass composition of claim 8, wherein the temperature for calcining was from about 900° C. to about 1150° C.

10. The bioactive glass composition of claim 8, wherein the temperature for calcining was from about 1000° C. to about 1150° C.

11. The bioactive glass composition of claim 1, wherein the reactant composition further comprises phosphoric acid.

12. The bioactive glass composition of claim 1, wherein the composition is used to form calcium phosphate.

13. The bioactive glass composition of claim 1, wherein the bioactive glass composition maintains a neutral pH as it degrades.

14. A method for treating injured or diseased skeletal muscle comprising contacting the injured or diseased skeletal muscle with an effective amount of the bioactive glass composition of claim 1.

15. The method of claim 14, wherein the injured or diseased skeletal muscle has an increase in average myofiber area after at least 8 days of treatment with the bioactive glass composition as compared to an injured or diseased skeletal muscle that undergoes an otherwise similar treatment with saline.

16. The method of claim 14, wherein the injured or diseased skeletal muscle has a lower embryonic myosin heavy chain (eMyHC) concentration after at least 5 days of treatment with the bioactive glass composition as compared to an injured or diseased skeletal muscle that undergoes an otherwise similar treatment with saline.

17. The method of claim 14, wherein the injured or diseased skeletal muscle has an increased muscle mass or increased muscle peak force after at least 10 days of treatment with the bioactive glass composition as compared to an injured or diseased skeletal muscle that undergoes an otherwise similar treatment with saline.

18. (canceled)

19. The method of claim 14, wherein the injured or diseased skeletal muscle has an increased angiogenesis after at least 5 days of treatment with the bioactive glass composition as compared to an injured or diseased skeletal muscle that undergoes an otherwise similar treatment with saline.

20. The method of claim 14, wherein the injured or diseased skeletal muscle is injured skeletal muscle and the injured skeletal muscle is a pulled muscle, traumatically injured muscle, ruptured muscle, injured muscle resulting from muscle overuse or misuse, or a combination thereof.

21. (canceled)

22. The method of claim 20, wherein the injured muscle resulting from muscle overuse or misuse is the result of a sports injury.

23. The method of claim 14, wherein the injured or diseased skeletal muscle is diseased skeletal muscle and the diseased skeletal muscle is dystrophic skeletal muscle, cachexic skeletal muscle, sarcopenic skeletal muscle, or a combination thereof.

24. (canceled)

25. A method for treating injured or diseased brain or nerve tissue comprising contacting the injured or diseased brain or nerve tissue with an effective amount of the bioactive glass composition of claim 1.
